(19) United States
(12) Patent Application Publication
(10) Pub. No.: US 2006/0069181 A1
(43) Pub. Date: Mar. 30, 2006

(54) Dental adhesive composition

(75) Inventors: Christoph Thalacker, Weilheim (DE); Andreas A. Heumann, Kaufering (DE)

Correspondence Address: 3M INNOVATIVE PROPERTIES COMPANY, PO BOX 33427, ST. PAUL, MN, 55133-3427, US

(73) Assignee: 3M ESPE AG

(21) Appl. No.: 11/234,451

(22) Filed: Sep. 23, 2005

(30) Foreign Application Priority Data
Sep. 24, 2004 (EP) 04022876.9

(51) Int. Cl. A61K 6/08

(52) U.S. Cl. 523/116

(57) ABSTRACT

The present invention relates to a dental composition with optimized viscosity and/or rheological behavior. The present invention provides a dental composition comprising: (d) at least one carboxylic acid functional polymer, (e) at least one acid derivative with stronger acidity than the carboxylic acid functional polymer (a), substituted with at least one polymerizable ethylenically unsaturated group, (f) 0.1 to 10 weight % of water,
wherein the carboxylic acid functional polymer (a) and the acid derivative (b) are present at least in an amount effective that the dental composition exhibit shear thinning and/or a viscosity from 1.0 to 20 Pa·s, when measured with a plate/plate geometry and at a shear rate of 0.5 to 1 s⁻¹. It further relates to the use of this dental composition and to a method for preparing a dental adhesive.

This application claims priority from European Application Serial No. 04022876.9, filed Sep. 24, 2004.

### FIELD OF INVENTION

The present invention relates to a dental adhesive composition. More specifically, it relates to a self-etching dental material comprising a carboxylic acid functional polymer. It can be used for adhering a dental restoration material to a tooth structure.

### BACKGROUND

The restoration of dental structures often involves the adhesive securing of a dental material to the hard tissue. Another use for adhesives in dentistry is bonding orthodontic appliances to teeth. As many dental materials like resin composites of the art due to their chemistry do not adhere to hard tissue like dentin or enamel, a pre-treatment is necessary. This includes etching of the enamel surface with inorganic or organic acids, followed by priming and subsequent application of a bonding agent. In many cases, between such steps, one or more rinsing and drying steps are used. As a result, dental restoration and the application of orthodontic appliances typically involve multi-step procedures.

To simplify conventional restorative and/or orthodontic procedures, for example, it is desirable to provide compositions that accomplish two or more of the steps of etching, priming and/or bonding. Many of the adhesive compositions known from the art combine two or more of these steps.

A further important prerequisite for a long-lasting adhesive bond between the dental structure and the restorative material or orthodontic appliance is the formation of a homogeneous adhesive film on the dental structure. Poor wetting of the tooth structure by the adhesive, or a too low viscosity of the adhesive can result in an inhomogeneous adhesive film or pooling of the adhesive. Thus, many adhesive compositions known from the state of the art try to overcome this problem of poor rheological properties and/or a low viscosity.

Many current dental adhesives contain different viscosity modifiers or rheology additives such as fumed silica or polymeric thickeners.

U.S. Pat. No. 4,952,613 (Kuraray) describes dental compositions comprising an organic carboxylic acid, a metal chloride and water. The dental composition could further comprise a thixotropic agent selected from the group consisting of high molecular weight thickeners of polyvinylpyrrolidone and carboxymethylcellulose, and a highly dispersable silica.

EP 1 287 805 A1 (GC) describes a one-pack type dental composition comprised of a polymerizable composition containing among others 1-5% by weight of a polymerizable monomer containing a phosphoric acid group, 10-40% by weight of a polymerizable monomer containing a plurality of carboxyl groups in one molecule and among others 15-50% by weight of water and a viscosity modifier having a mean particle size of 0.01-0.05 μm. Suitable viscosity modifiers are ultra fine inorganic fillers such as fumed silica, aluminum oxide and titanium dioxide or a glass powder.

U.S. Pat. No. 6,506,816 B1 (3M) describes dental resin cement materials having unique handling properties that comprise a filler, a polymerizable resin and a polymeric handling modifier which is dispersed in the polymerizable resin.

There are some materials that can exhibit a change in the Theological behavior after mixing of two or more components:

U.S. Pat. No. 4,533,701 (Tokuyama Soda) describes an adhesive coating material with high water resistance. The material comprises (1) a polymer including two carboxyl groups or one carboxylic anhydride group, and (2) an organic titanate. The carboxyl groups or the carboxylic anhydride group is bonded to adjacent carbon atoms of the polymer. Since the two carboxyl groups or one carboxylic anhydride group form a bridge of a high strength with the organic titanate compound, an especially high water resistance is given to this material. As known by a person skilled from the art the combination of these compounds can lead to gelation and to an increased viscosity of the material.

U.S. Pat. No. 4,648,844 (Kuraray) describes two pack type dental lining compositions comprising (a) a solution of a polymer having at least 10 mole % of a carboxyl-containing vinyl monomer, and (b) a solution of a zirconium chelate compound.

U.S. Pat. No. 6,583,197 B1 (Shofu) describes a dental adhesive composition comprising among others (a) a polymerizable unsaturated monomer containing 5% by weight of a radical polymerizable monomer having an acid group, and (b) an acid-reactive filler. An acid-base reaction between these compounds leads to a gradual increase in the viscosity.

U.S. Pat. No. 5,256,447 (3M) describes a method of adhering a restorative material to a substrate using an adhesive composition. The adhesive composition comprises an ethylenically unsaturated phosphorylated compound, a carboxylic acid functional polymer, and a curing agent. It is used to adhere an amalgam to hard tissue such as dentin or enamel. As a conventional adjuvant the composition can also contain viscosity modifiers. The disclosed composition does not contain any water.

U.S. Pat. No. 5,922,786 (3M) discloses a multiple-part dental adhesive primer composition that gives high adhesion values without the need for a separate acid etching step. The composition provides a Part A comprising an acidic polymerizable compound and a polymerizable diluent wherein the pH of Part A is greater than about 2. It further provides a Part B comprising an acidic material wherein the pH of Part B is below about 2. The composition contains 0.5 to 90% by weight of water in Part A or in Part B or in both parts.

The dental adhesives with rheology modifiers or thickeners described above suffer from several drawbacks: If particulate viscosity modifiers or rheology additives as fumed silica are used, upon standing of the composition, gravitational separation of the particles may occur. Moreover, they have to be dispersed in the liquid in an extra step rendering the production process more expensive. The other viscosity modifiers described above, e.g. polyvinylpyrrolidone and carboxymethylcellulose, often do not copolymerize with the other components of the formulation, which might lead to an impaired performance of the adhesive. Moreover, if the adhesive is prepared by mixing two or more compositions, as most of the known self-etching adhesives, the use of too viscous compositions can lead to dosing and mixing problems, and to the incorporation of air bubbles.

### SUMMARY OF THE INVENTION

This invention provides a dental composition comprising: (a) at least one carboxylic acid functional polymer, (b) at least one acid derivative with stronger acidity than the carboxylic acid functional polymer (a), substituted with at least one polymerizable ethylenically unsaturated group, (c) 0.1 to 10 weight % of water. The carboxylic acid functional polymer (a) and the acid derivative (b) are present at least in an amount effective that the dental composition exhibits shear thinning, and/or a viscosity of 1.0 to 20 Pa·s, when measured with a plate/plate geometry and at a shear rate of 0.5 to 1 s⁻¹. The methods of viscosity measurements are described below.

Such compositions can be used to provide improved adhesion between dental restorative material, and/or an orthodontic appliance to a dental substrate surfaces such as dentin, enamel, bone or other hard tissue. Additionally, the compositions can promote a self-etching adhesive with desirable rheological properties and/or viscosity while avoiding particulate viscosity modifers.

It has been found that compositions with a carboxylic acid functional polymer and which may be ethylenically unsaturated, an acid derivative (b) with stronger acidity than the carboxylic acid functional polymer, and which may be ethylenically unsaturated, and 0.1 to 10 weight % of water can be provided which show increased viscosities and/or shear thinning behavior compared to the compositions known from the art.

In a preferred embodiment, the invention relates to a dental composition, comprising: (a) 1 to 30 weight % of at least one carboxylic acid functional polymer, substituted with at least one polymerizable ethylenically unsaturated group, (b) 10 to 80 weight % of at least one acid derivative with stronger acidity than the carboxylic acid functional polymer (a), substituted with at least one polymerizable ethylenically unsaturated group, (c) 0.1 to 10 weight % of water, (d) 1 to 80 weight % of at least one unsaturated monomer and/or prepolymer, (e) 0.1 to 10 weight % of initiators and stabilizers, wherein the carboxylic acid functional polymer (a) and the acid derivative (b) are present at least in an amount effective that the dental composition exhibit shear thinning and/or a viscosity from 1.0 to 20 Pa·s, when measured with a plate/plate geometry and at a shear rate of 0.5 to 1 s⁻¹.

The described dental composition comprises 0.1 to 10 weight % of water. For higher water contents, the viscosity may become too low and the desired viscosity and/or shear thinning behaviors cannot readily be obtained. Preferably, the dental composition of the invention comprises 0.1 to 6 weight % of water.

Preferably, the dental composition of the invention shows a shear thinning behavior. Thus, the viscosity of the dental composition at a shear rate of 0.5 to 1 s⁻¹ is from 1.0 to 20 Pa·s and the viscosity at a shear rate of 1000 s⁻¹ is less than 1.0 Pa·s. Alternatively or additionally, the viscosity of the dental composition at a shear rate of 1000 s⁻¹ is less than 30% of the viscosity at a shear rate of 0.5 s⁻¹. A composition with shear thinning behavior like this embodiment of the present invention is advantageous as it exhibits better handling properties and it allows a uniform wetting of dental structures.

It is a further aspect of the invention that the composition in a preferred embodiment may not comprise any particulate viscosity modifiers such as fumed silica, aluminum oxide and titanium dioxide or a glass powder.

In a preferred embodiment, the dental composition comprises a carboxylic acid functional polymer (a) according to the following formula (I):
B(X)ₘ(Y)ₙ (I)
wherein B represents an organic backbone, each X independently is a carboxylic group, each Y independently is a polymerizable group, m is a number having an average value of 2 or more, and n is a number having an average value of 0 or more.
Preferably, the backbone B is an oligomeric or polymeric backbone of carbon-carbon bonds, optionally containing substituents which do not unduly interfere with the polymerization reaction, such as oxygen, nitrogen or sulfur heteroatoms. Suitable Y groups include, but are not limited to, substituted and unsubstituted acrylates, methacrylates, alkenes and acrylamides. The weight average molecular weight of the compound according the formula (I) is at least about 250, preferably between about 500 and 500,000 and more preferably between about 1,000 and 100,000.

Preferably, the carboxylic acid functional polymer (a) is selected from the group consisting of homopolymers and copolymers of unsaturated mono-, di- or tricarboxylic acids and/or their anhydrides optionally substituted with at least one ethylenically unsaturated group. This includes, for example, homopolymers of poly(meth)acrylic acid, copolymers of (meth)acrylic and itaconic acid, (meth)acrylic and maleic acid, methyl vinyl ether and maleic anhydride or maleic acid, ethylene and maleic anhydride or maleic acid, and styrene and maleic anhydride or maleic acid, each polymer having at least one acidic group substituted with a polymerizable ethylenically unsaturated group.

Examples of carboxylic acid functional polymers, optionally substituted with polymerizable ethylenically unsaturated groups according to component (a) include, but are not limited to homopolymers and copolymers of unsaturated mono-, di-, or tricarboxylic acids commonly used to prepare glass ionomer cements. Representative polyalkenoic acids are described, for example, in U.S. Pat. Nos. 3,655,605; 4,016,124; 4,089,830; 4,143,018; 4,342,677; 4,360,605 and 4,376,835. Particularly preferred polyalkenoic acids also include homopolymers of polyacrylic acid, and copolymers of acrylic and itaconic acids, acrylic and maleic acids, methyl vinyl ether and maleic anhydride or maleic acid, ethylene and maleic anhydride or maleic acid, and styrene and maleic anhydride or maleic acid. Particularly preferred carboxylic acid functional polymers substituted with polymerizable ethylenically unsaturated groups are (meth)acrylate functionalized copolymers of acrylic acid, (meth)acrylic acid, maleic acid, and itaconic acid as described e.g. in EP 0 323 120 B1. This document is explicitly mentioned and its disclosure, especially the disclosure relating to the preparation of (meth)acrylate functionalized copolymers of acrylic acid, (meth)acrylic acid, maleic acid, and itaconic acid disclosed in the above mentioned location, is regarded as being part of the disclosure of the present invention. Mixtures of such polymers can be used if desired.

The carboxylic acid functional polymer (a) is preferably present in an amount of from 2-15 parts by weight, preferably from 2-10 parts by weight, more preferably from 3-8 parts by weight.

The acid functionality of the acid derivative (b) may be selected from oxyacids or thio-oxy acids of B, C, N, S, P having a pKa value of less than 4.75. If desired, a precursor to the acid such as an acid anhydride, or ester can be used in place of the acid itself, e.g., to generate the desired acid in situ. Suitable polymerizable ethylenically unsaturated groups include, but are not limited to, substituted and unsubstituted acrylates, methacrylates, alkenes and acrylamides.

Examples of acid derivatives with stronger acidity than the carboxylic acid functional polymer (a), optionally substituted with one or more polymerizable ethylenically unsaturated groups according to component (b) include, but are not limited to 2-methacryloyloxyethyl phosphate, 2-methacryloyloxypropyl phosphate, 3-methacryloyloxypropyl phosphate, 2-methacryloyloxybutyl phosphate, 3-methacryloyloxybutyl phosphate, 4-methacryloyloxybutyl phosphate, 5-methacryloyloxy-3-oxa-pentyl phosphate, bis(2-methacryloyloxyethyl) phosphate, bis(2-methacryloyloxypropyl) phosphate, bis(3-methacryloyloxypropyl) phosphate, bis(2-methacryloyloxybutyl) phosphate, bis(3-methacryloyloxybutyl) phosphate, bis(4-methacryloyloxybutyl) phosphate, bis(5-methacryloyloxy-3-oxa-pentyl) phosphate, glycerol-1,3-dimethacrylate-2-phosphate, glycerol-1,2-dimethacrylate-3-phosphate, bis(glycerol-1,3-dimethacrylate) phosphate, bis(glycerol-1,2-dimethacrylate) phosphate, (glycerol-1,2-dimethacrylate)(glycerol-1,3-dimethacrylate) phosphate, (trimethylolpropane dimethacrylate) phosphate, bis(trimethylolpropane dimethacrylate) phosphate, (trimethylolethane dimethacrylate) phosphate, bis(trimethylolethane dimethacrylate) phosphate, pentaerythritol trimethacrylate phosphate, phosphoric acid, maleic acid, maleic acid monoesters such as mono(methacryloyloxyethyl)maleate, sulfuric acid, sulfoethyl methacrylate, sulfopropyl methacrylate, 2-acrylamido-2-methyl-propanesulfonic acid, dodecylbenzene sulfonic acid, 2-naphthalene sulfonic acid, trifluoroacetic acid, trifluoromethanesulfonic acid, and mixtures thereof.

The acid derivative (b) can be present in an amount of 10-80 parts by weight of the total composition. Preferably, (b) can be present in an amount of 20-80, more preferably 20-60 parts by weight.

The amount of (a) and (b) in the total composition can be 10-90 parts by weight, preferably 15-80 parts by weight, more preferably 20-70 parts by weight.

The dental composition of the invention optionally comprises 1 to 80 weight % of at least one unsaturated monomer and/or prepolymer.

Examples for these unsaturated monomers are ethylene glycol di(meth)acrylate, diethylene glycol di(meth)acrylate, triethylene glycol di(meth)acrylate, tetraethylene glycol di(meth)acrylate, polyethylene glycol di(meth)acrylate, 1,3-butylene glycol di(meth)acrylate, 1,6-hexane diol di(meth)acrylate, neopentyl glycol di(meth)acrylate, tripropylene glycol di(meth)acrylate, polypropylene glycol di(meth)acrylate, glycerol di(meth)acrylate, bisphenol A di(meth)acrylate, bisphenol A glycidyl di(meth)acrylate, bisphenol A propyl di(meth)acrylate, bisphenol A isopropyl di(meth)acrylate, ethylene oxide modified bisphenol A di(meth)acrylate, ethylene oxide modified bisphenol A glycidyl di(meth)acrylate, 2,2-bis(4-methacryloxypropoxyphenyl) propane, 7,7,9-trimethyl-4,13-dioxy-3,14-dioxa-5,12-diazahexadecane-1,16-diol di(meth)acrylate, neopentyl glycol hydroxypivalic acid ester di(meth)acrylate, caprolactone modified hydroxypivalic acid neopentyl glycol ester di(meth)-acrylate, trimethylol ethane di(meth)acrylate, trimethylol propane di(meth)acrylate, trimethylol methane tri(meth)acrylate, trimethylol ethane tri(meth)acrylate, trimethylol propane tri(meth)acrylate, pentaerythritol tri(meth)acrylate, dipentaerythritol tri(meth)acrylate, pentaerythritol tetra(meth)acrylate, dipentaerythritol tetra(meth)acrylate, dipentaerythritol hexa(meth)acrylate, the reaction product of 3-chloro-2-hydroxypropyl (meth)acrylate and methylcyclohexane diisocyanate, the reaction product of 2-hydroxypropyl (meth)acrylate and methylcyclohexane diisocyanate, the reaction product of 2-hydroxypropyl (meth)acrylate and methylene bis (4-cyclohexylisocyanate), the reaction product of 2-hydroxypropyl(meth)acrylate and trimethylhexamethylene diisocyanate, the reaction product of 2-hydroxyethyl (meth)acrylate and isophorone diisocyanate, and the reaction product of 3-chloro-2-hydroxypropyl (meth)acrylate and isophorone diisocyanate, methyl (meth)acrylate, ethyl (meth)acrylate, propyl methacrylate, isopropyl methacrylate, n-butyl (meth)acrylate, iso-butyl (meth)acrylate, 2-ethylhexyl (meth)acrylate, lauryl (meth)acrylate, tridecyl (meth)acrylate, stearyl (meth)acrylate, cyclohexyl (meth)acrylate, benzyl (meth)acrylate, 2-hydroxyethyl (meth)acrylate, 2-hydroxypropyl (meth)acrylate, glycidyl (meth)acrylate, tetrahydrofurfuryl (meth)acrylate, allyl (meth)acrylate, 2-ethoxyethyl (meth)acrylate, methoxydiethylene glycol (meth)acrylate, methoxytetraethylene glycol (meth)acrylate, methoxypolyethylene glycol (meth)acrylate, phenoxy-diethyleneglycol (meth)acrylate, phenoxyhexaethyleneglycol (meth)acrylate, glycerol (meth)acrylate, tetrahydrofurfuryl (meth)acrylate, dicyclopentenyl (meth)acrylate, isobornyl (meth)acrylate, phenyl (meth)acrylate, pentaerythritol mono(meth)acrylate, dipentaerythritol mono(meth)acrylate, and mixtures thereof.

Examples of prepolymers are especially described in WO 01/44338 A1. This document is explicitly mentioned also and its disclosure, especially the disclosure relating to the preparation of unsaturated urethane prepolymers disclosed in the above mentioned location, is regarded as being part of the disclosure of the present invention and is hereby incorporated by reference. The prepolymers preferably do not contain hydroxy, acidic or ionic groups.

Another particularly preferred unsaturated prepolymer is an urethane functionalized polymer. This can be obtained, for example, by reaction of (A) 15 to 85 wt.-% of one or more α,ω-terminated poly(meth)acrylate diols, (B) 0 to 30 wt.-% of one or more radically curable, polyhydroxy-functional compounds, (C) 14 to 60 wt.-% of one or more polyisocyanates, (D) 1 to 40 wt. % of a monofunctional compound, reactive vis-à-vis isocyanate groups, which contain one or more radically curable groupings.

The prepolymers can have an average molecular weight (Mw) according to GPC measurements (against polystyrene standards) in the range of between 400 and 200,000 g/mol, preferably between 500 and 100,000 g/mol and more preferably between 600 and 20,000 g/mol. An example of such prepolymer is the step-growth polymerization product of Tego® Diol BD-1000, 2,4,4-trimethylhexyl-1,6-diisocyanate and 2-hydroxyethylmethacrylate. This is commercially available from Tego Chemie Service GmbH, Germany.

The unsaturated monomers and/or prepolymers can be present in an amount from 1-80 parts by weight, preferably from 10-50 parts by weight, and more preferably from 20-45 parts by weight,

Moreover, it has been found that adding urethane or amide group containing monomers, prepolymers, and/or polymers to the dental composition of the invention can lead to preferred compositions with improved properties like miscibility or wetting of a dental structure.

Initiators according to the invention can include curing agents like photoinitiators or photoinitiator systems for free radical polymerization known to the person skilled in the art.

Suitable photoinitiators for polymerizing free-radically photopolymerizable compositions include the class of phosphine oxides. Preferred phosphine oxide free radical initiators are acyl and bisacyl phosphine oxides such as those described in U.S. Pat. Nos. 4,298,738; 4,324,744; 4,385,109; 4,710,523; and 4,737,593; 6,251,963; and EP Application No. 0 173 567 A2.

Commercially available phosphine oxide photoinitiators capable of free-radical initiation when irradiated at wavelength ranges of greater than 380 nm to 450 nm include, bis(2,4,6-trimethylbenzoyl)phenyl phosphine oxide (IRGACURE 819, Ciba Specialty Chemicals), bis(2,6-dimethoxybenzoyl)-(2,4,4-trimethylpentyl) phosphine oxide (CGI 403, Ciba Specialty Chemicals), a 25:75 mixture, by weight, of bis(2,6-dimethoxybenzoyl)-2,4,4-trimethylpentyl phosphine oxide and 2-hydroxy-2-methyl-1-phenylpropan-1-one (IRGACURE 1700, Ciba Specialty Chemicals), a 1:1 mixture, by weight, of bis(2,4,6-trimethylbenzoyl)phenyl phosphine oxide and 2-hydroxy-2-methyl-1-phenylpropane-1-one (DAROCUR 4265, Ciba Specialty Chemicals), and ethyl 2,4,6-trimethylbenzylphenyl phosphinate (LUCIRIN LR8893X, BASF).

Other typical examples are combinations of photoinitiators or photoinitiator systems for free radical polymerization and include combinations of a sensitizing agent with a reducing agent.

As the sensitizing agent, those which can polymerize the polymerizable monomer by the action of a visible light having a wavelength of from 390 nm to 830 nm are preferred. Examples thereof include camphorquinone, benzil, furil, 3,3,6,6-tetramethylcyclohexanedione, diacetyl, benzyl dimethyl ketal, benzyl diethyl ketal, benzyl di(2-methoxyethyl) ketal, 4,4,′-dimethylbenzyl dimethyl ketal, anthraquinone, phenanthraquinone, 1-chloroanthraquinone, 2-chloroanthraquinone, 1,2-benzanthraquinone, 1-hydroxyanthraquinone, 1-methylanthraquinone, 2-ethylanthraquinone, 1-bromoanthraquinone, thioxanthone, 2-isopropyl thioxanthone, 2-nitrothioxanthone, 2-methyl thioxanthone, 2,4-dimethyl thioxanthone, 2,4-diethyl thioxanthone, 2,4-diisopropyl thio-xanthone, 2-chloro-7-trifluoromethyl thioxanthone, thioxanthone-10,10-dioxide, thio-xanthone-10-oxide, benzoin methyl ether, benzoin ethyl ether, isopropyl ether, benzoin isobutyl ether, benzophenone, bis(4-dimethylaminophenyl)ketone, 4,4,′-bisdiethylaminobenzophenone, acyl phosphine oxides such as (2,4,6-trimethylbenzoyl)diphenylphosphine oxide, and azide-containing compounds. These compounds may be used singly or in admixture.

As the reducing agent, tertiary amines and the like are generally used. Suitable examples of the tertiary amines include N,N-dimethyl-p-toluidine, N,N-dimethylaminoethyl methacrylate, triethanolamine, methyl 4-dimethylaminobenzoate, ethyl 4-dimethylaminobenzoate, and isoamyl 4-dimethylaminobenzoate. Examples of other reducing agents include, sodium sulfinate derivatives and organometallic compounds can also be used. These compounds may be used singly or in admixture. If desired, a combination of an initiator system including a sensitizer and a reducing agent with a phosphine oxide initiator can be used.

Further examples of suitable initiator systems can be found in the literature, e.g. J.-P. Fouassier, “Photoinitiation, Photopolymerization, and Photocuring”, Hanser Publishers, Munich, Vienna, N.Y., 1995, and J.-P. Fouassier, J. F. Rabek (eds.), “Radiation Curing in Polymer Science and Technology, Vol. 11”, Elsevier Applied Science, London, N.Y., 1993.

Moreover, ternary photopolymerization initiating systems consisting of a sensitizer, an electron donor and an onium salt such as those as described in U.S. Pat. No. 6,187,833; U.S. Pat. No. 6,025,406; U.S. Pat. No. 6,043,295; U.S. Pat. No. 5,998,495; U.S. Pat. No. 6,084,004 and U.S. patent application Ser. No. 10/050,218 can be used and the disclosure of the same are herein included by reference.

The initiators can be present in an amount of from 0.1-10 parts by weight, preferably from 0.2-10 parts by weight, more preferably from 0.3-5 parts by weight, based upon total composition weight.

Examples of stabilizers according to the invention are butylated hydroxytoluene (BHT), hydroquinone, hydroquinone monomethyl ether (MEHQ), 3,5-di-tert-butyl-4-hydroxyanisole (2,6-di-tert-butyl-4-ethoxyphenol), 2,6-di-tert-butyl-4-(dimethylamino)methylphenol or 2,5-di-tert-butyl hydroquinone, 2-(2′-hydroxy-5′-methylphenyl)-2H-benzotriazole, 2-(2′-hydroxy-5′-t-octylphenyl)-2H-benzotriazole, 2-hydroxy-4-methoxybenzophenone (UV-9), 2-(2′-hydroxy-4′,6′-di-tert-pentylphenyl)-2H-benzotriazole, 2-hydroxy-4-n-octoxybenzophenone, and 2-(2′-hydroxy-5′-methacryloxyethylphenyl)-2H-benzotriazole. Such adjuvants may optionally comprise reactive functionality so that they will be copolymerized with the resin.

Preferably, the stabilizers are present in an amount of from 0-5 parts by weight, more preferably from 0.001-2 parts by weight, most preferably from 0.01-1 parts by weight.

The dental composition of the invention may further contain commonly used auxiliaries like, for example, surfactants, solvents, fluoride releasing agents, non reactive inorganic fillers and photobleachable colorants.

Dental compositions of the present invention optionally contain surfactants including nonionic surfactants, anionic surfactants, cationic surfactants, amphoteric surfactants, and combinations thereof. Surfactants useful in compositions and methods of the present invention include non-polymerizable and polymerizable surfactants.

The nonionic surfactants are usually condensation products of an organic aliphatic or alkylaromatic hydrophobic compound and an alkylene oxide, such as ethylene oxide, which is hydrophilic. Almost any hydrophobic compound having a carboxy, hydroxy, amido, or amino group with a free hydrogen present can be condensed with ethylene oxide to form a nonionic surfactant.

Particularly suitable nonreactive, nonionic surfactants include, but are not limited to, those selected from the group consisting of the condensation products of a higher aliphatic alcohol, such as a fatty alcohol, containing about 8 to about 20 carbon atoms, in a straight or branched chain configuration, condensed with about 3 to about 100 moles, preferably about 5 to about 50 moles, most preferably about 5 to about 40 moles of ethylene oxide. Examples of such non-ionic, ethoxylated, fatty alcohol surfactants are the Tergitol 15-S series from Dow and Brij surfactants from Uniqema. Brij 35 Surfactant is Polyoxyethylene(23) lauryl ether.

Other suitable nonreactive nonionic surfactants include, but are not limited to, those selected from the group consisting of the polyethylene oxide condensates of one moles of alkyl phenol containing from about 6 to 12 carbon atoms in a straight or branched chain configuration, with about 3 to about 100 moles, preferably about 5 to about 50 moles, most preferably about 5 to about 40 moles of ethylene oxide. Examples of such nonreactive, nonionic surfactants are the Tergitol NP and Triton X series from Dow and Igepal CO and CA series from Rhodia. Tergitol NP and Igepal CO surfactants include nonylphenoxy poly(ethyleneoxy) ethanols. Triton X and Igepal CA surfactants include octylphenoxy poly(ethyleneoxy) ethanols.

Another group of usable, nonreactive, nonionic surfactants include, but are not limited to, those selected from the group consisting of block copolymers of ethylene oxide and propylene oxide or butylene oxide. Examples of such nonionic block copolymer surfactants are the Pluronic and Tetronic series of surfactants from BASF, and the Synperonic series of surfactants from Uniqema.

Still other useful, nonreactive, nonionic surfactants include but are not limited to those selected from the group consisting of sorbitan fatty acid esters, polyoxyethylene sorbitan fatty acid esters and polyoxyethylene stearates. Examples of such fatty acid ester nonionic surfactants are the Span, Tween, and Myrj surfactants from Uniqema. Span surfactants include C₁₂-C₁₈ sorbitan monoesters. Tween surfactants include poly(ethylene oxide) C₁₂-C₁₈ sorbitan monoesters. Myrj surfactants include poly(ethylene oxide) stearates.

Useful polymerizable, non-ionic surfactants typically have polymerizable groups such as hydroxyethyl methacrylate, hydroxypropyl methacrylate, glycerol dimethacrylate, pentaerythritol triacrylate and the like that are attached (e.g., via a urethane linkage) to a hydrocarbon group (e.g., hexyl, cyclohexyl, pentyl, or octyl) to a polyethylenegycol chain. The methacrylate or acrylate moiety (hereafter written as (meth)acrylate) together with the hydrocarbon group moiety form a non-polar hydrophobic end, while the polyethylene glycol chain forms a polar hydrophilic end. By varying the number of (meth)acrylate groups and the length or number of the polyethylene glycol or chains, surfactants with a wide variety of properties may be produced. In addition to the (meth)acrylate groups, other non-polar groups, such as fatty acids, may also be incorporated to provide an even more hydrophobic end, as desired. The polyethylene glycol chain may further be broken into several short chains if desired.

Useful polymerizable, nonionic surfactants include, for example, the polymerizable nonionic surfactants available under the Blemmer series from Nippon Oil and Fat, and the Noigen series from Dai-Ichi Kogyo Seiyaku.

Useful anionic surfactants include, for example, aliphatic metal carboxylates such as sodium laurate, sodium stearate and sodium oleate; sulfated aliphatic metal carboxylates such as sodium dioctylsulfosuccinic acid; metal salts of higher alcohol sulfates such as sodium dodecylsulfate, sodium lauryl sulfate, sodium cetyl sulfate, sodium stearyl sulfate and sodium oleyl sulfate; metal salts of higher alkylether sulfates such as sodium lauryl ether sulfate obtained by sulfating an ethylene oxide adduct with a lauryl alcohol; metal salts of alkylbenzenesulfonic acids such as sodium dodecylbenzenesulfonate; metal salts of alpha-olefinsulfonic acids synthesized by reacting sulfuric acid with an alpha-olefin; Igepon T obtained by reacting N-methyl taurine and oleic acid chloride; sulfosuccinic acid diesters typified by Aerosol OT; phosphoric acid ester salts of adducts of ethylene oxide with higher alcohols; and dithiophosphoric acid ester salts.

Useful cationic surfactants include, but are not limited to adducts of ethylene oxide with higher alkylamines such as stearylamine; amines prepared from lower amines; alkyltrimethyl ammonium salts such as lauryl trimethylammonium chloride; quaternary ammonium salts prepared from higher alkylamines typified by alkyldimethylbenzyl ammonium salts such as lauryl dimethylbenzyl ammonium chloride; Sapamine type quaternary ammonium salts; and quaternary ammonium salts prepared from lower amines typified by pyridinium salts such as Zelan AP and Velan PF.

Examples of polymerizable cationic surfactants include, but are not limited to, methacryloyloxyalkyl ammonium salts, such as, for example, methacryloyloxyethyl trimethylammonium chloride.

Examples of amphoteric surfactants can include metal salts of higher alkylaminopropionic acids such as sodium laurylaminopropionate and sodium stearylaminopropionate; and betaines such as lauryl dimethylbetaine, stearyl dimethylbetaine and lauryl dihydroxyethylbetaine.

The surfactants may be present in an amount of from 0.1-20 parts by weight, preferably from 0.2-10 parts by weight, more preferably from 0.3-5 parts by weight.

A preferred embodiment of the present invention includes a solvent. Examples of solvents include, but are not limited to, linear, branched or cyclic, saturated or unsaturated alcohols with 2 to 10 C atoms, ketones, esters or mixtures of two or more of said type of solvents. Especially preferred alcoholic solvents include methanol, ethanol, iso-propanol and n-propanol. Other suitable organic solvents include THF, acetone, methylethyl ketone, cyclohexanol, toluene, alkanes and acetic acid alkyl esters, and in particular, acetic acid ethyl ester. Generally, it is possible to use the above-mentioned solvents alone or as a mixture of two or more provided the solvent mixtures do not impair the adhesive properties, the viscosity and/or the rheological properties to such an extent that the desired results cannot be obtained. The solvent can be present in an amount from 0-20 parts by weight, preferably from 1-15 parts by weight, more preferably from 3-10 parts by weight,

The dental composition may further comprise a fluoride release agent. Examples of useful fluoride release agent include naturally occuring or synthetic fluoride minerals such as sodium fluoride; fluoride glass, such as fluoroaluminosilicate glass; simple and complex inorganic fluoride salts such as potassium zinc fluoride and potassium hexafluorotitanate; simple and complex organic fluoride salts, such as tetra ethyl ammonium tetra fluoroborate; or combinations thereof. Optionally, these fluoride sources can be treated with surface treatment agents. Optionally, the fluoride release agent can be present in an amount of from 0-20 parts by weight, preferably from 0.2-10 parts by weight, more preferably from 0.3-5 parts by weight.

Further, a non-reactive, inorganic filler may be present. Examples of a nonreactive inorganic filler include naturally-occurring or synthetic materials such as quartz, nitrides (e.g., silicon nitride), glasses derived from, for example Ce, Sb, Sn, Zr, Sr, Ba or Al, colloidal silica, feldspar, borosilicate glass, kaolin, talc, titania, and zinc glass, zirconia-silica fillers; low Mohs hardness fillers such as those described in U.S. Pat. No. 4,695,251; and submicron silica particles (e.g., pyrogenic silicas such as the “Aerosil” Series “OX 50”, “130”, “150” and “200” silicas sold by Degussa, and “Cab-O-Sil M5” silica sold by Cabot Corp.). Preferred filler particles are quartz, submicron silica, and non-vitreous microparticles of the type described in U.S. Pat. No. 4,503,169. Mixtures of these fillers are also contemplated, as well as combination fillers made from organic and inorganic materials.

Optionally, the surface of the filler particles may be treated with a surface treatment, such as a silane coupling agent, in order to enhance the bond between the filler and the polymerizable resin. The coupling agent may be functionalized with reactive curing groups, such as acrylates and/or methacrylates.

The non-reactive inorganic filler can be present in an amount of from 0-70 parts by weight, preferably from 5-60 parts by weight, more preferably from 10-50 parts by weight.

In certain instances, the fluoride source of the fluoride release agent and the filler can be the same.

Optionally, a photobleachable colorant such as, for example, Rose Bengal, Methylene Violet, Methylene Blue, Fluorescein, Eosin Yellow, Eosin Y, Ethyl Eosin, Eosin bluish, Eosin B, Erythrosin B, Erythrosin Yellowish Blend, Toluidine Blue, 4′,5′-Dibromofluorescein and blends thereof may be present. Further examples of photobleachable colorants can be found in U.S. Pat. No. 6,444,725 and such disclosure is herein incorporated by reference. The color of the compositions of the present invention may be additionally imparted by a sensitizing compound. The amount of the photobleachable colorant can be from 0-5 parts by weight, preferably from 0.05-3 parts by weight, more preferably from 0.1-2 parts by weight.

The optional components mentioned above might be present in the composition alone or in combination with the other optional components.

In another preferred embodiment, the composition of the present invention is provided as a kit of at least two parts, A and B, wherein one part A comprises
(a) at least one carboxylic acid functional polymer, and
(aa) 0 to 10 weight % of water,
   and wherein another part B comprises
(b) at least one acid derivative with stronger acidity than the carboxylic acid functional polymer (a), substituted with at least one polymerizable ethylenically unsaturated group, and
(bb) less than 0.5 weight % of water,
   wherein the carboxylic acid functional polymer (a) and the acid derivative (b) are present at least in an amount effective that the mixture of the parts exhibit shear thinning and/or a viscosity that is higher than that of each of the separate parts, when measured with a plate/plate geometry and at a shear rate of 0.5 to 1 s⁻¹.

Preferably, the kit is provided with two parts, part A and part B provided in a ratio of A:B from 5:1 to 1:5, and preferably in a ratio of A:B of 1:4.

It is advantageous to mix two or more low viscosity compositions to obtain a more viscous composition, which ideally exhibits shear thinning. In this case, ease of dosing, mixing, and application lead to a more robust, user friendly system.

The dental compositions of the invention can be used as dental adhesives, especially for securing dental filling materials, as self-adhesive dental pit and fissure sealants, as self-adhesive dental desensitizers, and as self-adhesive dental restorative materials

The dental compositions may be used for adhering of dental fillings based on methacrylate like materials described in the prior art e.g. in WO 01/30305 A1, WO 01/30306 A1, WO 01/30307 A1.

Surprisingly it has been found that the dental composition of the present invention can be used to secure dental filling materials based on cationically polymerizable resins to tooth or bone. Especially for cationically polymerizable resins like the epoxy resins and the silicon containing and polysiloxane epoxides an adhesive system was needed.

Suitable dental filling materials based on cationically polymerizable resins are described e.g. in U.S. Pat. No. 6,254,828, U.S. Pat. No. 6,084,004, WO 01/51540 A1, WO 02/66535 A1, WO 98/47046 A1, WO 98/47047 A1. The above mentioned documents are explicitly mentioned, and their disclosure is herein incorporated by reference.

The dental compositions of the present invention can provide at least comparable adhesion values to state of the art, self-adhesive dental materials. In addition, they exhibit an optimized rheological behavior (e.g. shear thinning) and/or an increased viscosity, to provide better handling, and, if applied to adhesively secure dental filling materials, a more homogeneous adhesive film can be provided without pooling or dewetting effects. Moreover, these effects are provided without the need of adding a particulate viscosity modifier (e.g. fumed silica). Thus, on the one hand, costly production steps like dispersing these particulate viscosity modifiers can be avoided, and gravitational separation of the composition upon standing can be eliminated. If the dental compositions of the present invention are provided as a kit of at least two parts, the composition of the desired Theological behavior and/or viscosity can be obtained by mixing two or more parts of lower viscosity thus providing clear advantages in terms of dosing, mixing, and application.

The inventive compositions are usually applied to the tooth surface in an amount sufficient to etch and prime the dental tissue. In this respect, the following steps can be followed:
a) applying the composition to the surface of a tooth (enamel and/or dentin), preferably using a brush or a sponge,
b) optionally dispersing the composition to form a thin film, preferably using a stream of air,
c) light or redox initiated curing of the composition,
d) optionally, applying a dental filling composition adjacent the composition.
   Optionally, one or more of the steps mentioned above can be repeated.

The composition can generally be provided in any type of package, e.g. tubes, or flasks. For the application of small amounts of liquids, however, the prior art discloses a number of alternatives which facilitate the application. For example, for dental application multi-chamber packaging devices such as those described in EP 0 895 943 B1, WO-02/38468 A1, WO 01/58869 A1 are preferred.

If the dental composition is provided in at least two parts, the partitioning of the components into the parts should prevent undesired reactions of the composition. Components comprising OH groups should preferably stored in one part, the acid derivatives (b) with stronger acidity than the carboxylic acid functional polymer in the other part.

The composition of the present invention is usually prepared by mixing the components, e.g. by stirring or shaking.

The enamel adhesion of the inventive dental composition is usually in the range of from 2-40 MPa, preferably in the range of from 5-35 MPa, more preferably in the range of from 10-30 MPa, measured as described below.

The dentin adhesion of the inventive dental composition is usually in the range of from 2-40 MPa, preferably in the range of from 5-35 MPa, more preferably in the range of from 10-30 MPa, measured as described below.

The viscosity of the inventive dental composition at a shear rate of 0.5 s⁻¹ is usually in the range of from 1-20 Pa·s, preferably in the range of from 1-15 Pa·s, measured as described below. If the composition is provided as a kit of at least two parts, the viscosity of the mixture of the parts is preferably equal or higher than the viscosities of the parts alone. The viscosity vs. shear rate behavior of the inventive dental composition preferably exhibits shear thinning, i.e. decreasing viscosity with increasing shear rate.

### EXAMPLES

The invention is further described by the following examples. The examples are for illustrative purpose only, and the particular materials and amounts thereof recited in these examples, as well as other conditions and details, should not be construed to unduly limit this invention. Unless otherwise indicated, all parts and percentages are on a weight basis, all water is deionized water, and all molecular weights are weight average molecular weight.

Abbreviations:

PM2 mixture of reaction products of 2-hydroxyethyl methacrylate (HEMA) with phosphorus pentoxide, commercially available from Nippon Kayaku as Kayamer PM2
PGDMA mixture of reaction products of glycerol di(methacrylate) (GDMA) with phosphorus pentoxide
DBSA dodecylbenzene sulfonic acid
SEMA 2-sulfoethyl methacrylate
MEMA mono(methacryloyloxyethyl)maleate
AA glacial acetic acid
HEMA hydroxyethyl methacrylate
TEGDMA triethylene glycol dimethacrylate
TMHDI 2,4,4-trimethylhexyl-1,6-diisocyanate
PUMA step growth polymerization product of Tego® Diol BD-1000, TMHDI, and HEMA (see preparative example 2 of WO 01/44338 A1)
BisEMA ethoxylated bisphenol A dimethacrylate (with a mean of 4 ethylene oxide units per molecule)
BPDMA bisphenol A dipropylmethacrylate
VBP the precipitated dry polymer of example 11 of EP 0 323 120 B 1
CPQ camphorquinone
EDMAB ethyl 4-dimethylaminobenzoate
DPIPF6 diphenyliodonium hexafluorophosphate
Brij 35 polyoxyethylene(23) lauryl ether
MEHQ hydroquinone monomethyl ether
BHT butylated hydroxytoluene
   Test Methods
   Adhesion Shear Bond Strength to Enamel or Dentin Test Method

Adhesive shear bond strength to enamel or dentin for a given test sample was evaluated by the following procedure.

Preparation of Teeth. Bovine incisal teeth, free of soft tissue, were embedded in circular acrylic disks. The embedded teeth were stored in water in a refrigerator prior to use. In preparation for adhesive testing, the embedded teeth were ground to expose a flat enamel or dentin surface using 120-grit sandpaper mounted on a lapidary wheel. Further grinding and polishing of the tooth surface was done manually using 320-grit sandpaper. The teeth were continuously rinsed with water during the grinding process. The polished teeth were stored in deionized water and used for testing within 2 hours after polishing. The teeth were allowed to warm in a 36° C. oven to between room temperature (23° C.) and 36° C. before use.

Teeth Treatment. An adhesive test sample was applied with a dental applicator brush over the entire surface of the prepared enamel or dentin surface and rubbed in with moderate finger pressure for 20 seconds. Then a stream of compressed air was blown on until no more moving liquid could be seen. After light curing for 10 seconds with a dental curing light (Elipar™ Trilight, 3M ESPE AG, Seefeld, Germany, light intensity: approx. 800 mW/cm²), the applicator brush was rewetted with the adhesive, and a second layer of adhesive was applied without rubbing in. This layer was thinned for 2 seconds only with a gentle stream of air. Then, the adhesive coating was light cured again for 10 seconds with an Elipar™ Trilight dental curing light. A 2.5 mm thick Teflon mold with a hole approximately 4.7 mm in diameter was clamped to the embedded tooth such that the hole in the mold exposed part of the adhesively prepared tooth surface. A composite material, i.e. an epoxy-based filling material as described in WO 01/10388, page 12, or A3 shade of FILTEK™ Z250 Universal Restorative (3M ESPE Dental Products, St. Paul, Minn.), was filled into the hole such that the hole was completely filled, but not overfilled, and light cured for 40 seconds in the case of the epoxy based filling material, or 20 seconds in the case of the FILTEK Z250 material to form a “button” that was adhesively attached to the tooth.

Adhesive Bond Strength Testing. The adhesive strength of a cured test example was evaluated by mounting the assembly (described above) in a holder clamped in the jaws of Zwick Universal testing machine (Zwick Z010, Zwick GmbH, Ulm, Germany) with the polished tooth surface oriented parallel to the direction of pull. A loop of orthodontic wire (0.71 mm diameter) was placed around the composite button adjacent to the polished tooth surface. The ends of the orthodontic wire were clamped in the pulling jaw of the Zwick apparatus and pulled at a crosshead speed of 2 mm/min, thereby placing the adhesive bond in shear stress. The force in Newtons (N) at which the bond failed was recorded, and this number was converted to a force per unit area (MPa) using the known surface area of the button. Each reported value of adhesion to enamel or adhesion to dentin represents the average of 5 replicates.

Rheology Measurements

Viscosity vs Shear Rate Curve. The viscosity was measured using a Bohlin CVO 120 rheometer (Bohlin Instruments, NJ) with a plate/plate geometry under controlled shear rate at 23° C. The plate diameter was 40 mm, and the separation between the plates 200 μm. The shear rate was ramped up from 0.5 to 1000 s⁻¹ and in reverse rate down to 0.5 from 1000 s⁻¹. This shear rate ramping took place every 10 seconds, with a 10 second delay time and 10 s integration time, for a total of 20 logarithmically-spaced shear rate steps per run (a run being defined as both increasing and decreasing shear direction).

### Examples 1-43

For a dental composition consisting of part A and part B two solutions A and B were prepared. Solution A consists of HEMA (30 parts), water (30 parts), VBP (30.1 parts), DPIPF6 (4.9 parts), Brij 35 (4.9 parts), BHT (0.1 parts). Solutions B1-43 were prepared consisting of an acid (b) (amount according to Table 1), BisEMA (amount according to Table 1), CPQ (1.25 parts), EDMAB (2 parts), and BHT (0.125 parts).

Both parts were mixed together at a ratio of 1.000 gram of solutions B1-43 to 0.250 grams of solution A. As a test for the handling properties and the viscosity of the compositions the resulting mixtures were checked visually for gel formation. The formation of a gel by mixing the two parts A and B of the dental composition indicates an increase in viscosity and improved handling properties. In Table 1, “−” denotes no visible gel formation, “+” denotes a visible gel formation, and “++” denotes a strong gel formation. The formulations with the desirable viscosity and rheological properties are among those denoted with a “+”.

### Comparative Examples 44-47

As comparative examples four mixtures with same ratio of solutions B44-47 and solution A were prepared containing the same ingredients for solution A. Solutions B44-47 consist of glacial acetic acid as acid (b) (amount according to Table 1), BisEMA (according to Table 1), CPQ (1.25 parts), EDMAB (2 parts), and BHT (0.125 parts).

TABLE 1

| Example | | | acid (b) | BisEMA | gel |
|---|---|---|---|---|---|
| no. | Solution B | acid (b) | (parts) | (parts) | formation |
| 1 | B1 | PM2 | 10,000 | 86,625 | − |
| 2 | B2 | PM2 | 20,000 | 76,625 | + |
| 3 | B3 | PM2 | 30,000 | 66,625 | + |
| 4 | B4 | PM2 | 40,000 | 56,625 | + |
| 5 | B5 | PM2 | 50,000 | 46,625 | + |
| 6 | B6 | PM2 | 60,000 | 36,625 | + |
| 7 | B7 | PM2 | 70,000 | 26,625 | + |
| 8 | B8 | PM2 | 80,000 | 16,625 | + |
| 9 | B9 | PM2 | 90,000 | 6,625 | ++ |
| 10 | B10 | PM2 | 96,625 | 0,000 | ++ |
| 11 | B11 | PGDMA | 10,000 | 86,625 | − |
| 12 | B12 | PGDMA | 20,000 | 76,625 | + |
| 13 | B13 | PGDMA | 30,000 | 66,625 | + |
| 14 | B14 | PGDMA | 40,000 | 56,625 | + |
| 15 | B15 | PGDMA | 50,000 | 46,625 | + |
| 16 | B16 | PGDMA | 60,000 | 36,625 | + |
| 17 | B17 | PGDMA | 70,000 | 26,625 | ++ |
| 18 | B18 | PGDMA | 80,000 | 16,625 | ++ |
| 19 | B19 | PGDMA | 90,000 | 6,625 | ++ |
| 20 | B20 | PGDMA | 96,625 | 0,000 | ++ |
| 21 | B21 | DBSA | 20,000 | 76,625 | + |
| 22 | B22 | DBSA | 40,000 | 56,625 | ++ |
| 23 | B23 | DBSA | 60,000 | 36,625 | ++ |
| 24 | B24 | SEMA | 10,000 | 86,625 | − |
| 25 | B25 | SEMA | 20,000 | 76,625 | − |
| 26 | B26 | SEMA | 30,000 | 66,625 | + |
| 27 | B27 | SEMA | 40,000 | 56,625 | + |
| 28 | B28 | SEMA | 50,000 | 46,625 | ++ |
| 29 | B29 | SEMA | 60,000 | 36,625 | ++ |
| 30 | B30 | SEMA | 70,000 | 26,625 | ++ |
| 31 | B31 | SEMA | 80,000 | 16,625 | ++ |
| 32 | B32 | SEMA | 90,000 | 6,625 | ++ |
| 33 | B33 | SEMA | 96,625 | 0,000 | ++ |
| 34 | B34 | MEMA | 10,000 | 86,625 | − |
| 35 | B35 | MEMA | 20,000 | 76,625 | − |
| 36 | B36 | MEMA | 30,000 | 66,625 | − |
| 37 | B37 | MEMA | 40,000 | 56,625 | + |
| 38 | B38 | MEMA | 50,000 | 46,625 | + |
| 39 | B39 | MEMA | 60,000 | 36,625 | + |
| 40 | B40 | MEMA | 70,000 | 26,625 | + |
| 41 | B41 | MEMA | 80,000 | 16,625 | ++ |
| 42 | B42 | MEMA | 90,000 | 6,625 | ++ |
| 43 | B43 | MEMA | 96,625 | 0,000 | ++ |
| 44 comp. | B44 | AA | 20,000 | 76,625 | − |
| 45 comp. | B45 | AA | 40,000 | 56,625 | − |
| 46 comp. | B46 | AA | 60,000 | 36,625 | − |
| 47 comp. | B47 | AA | 80,000 | 16,625 | − |

As can be seen for examples 1-43, the addition of an acid derivative with a stronger acidity than the carboxylic acid functional polymer to Part B leads to an increased viscosity and gel formation of the mixtures. Preferably the acid derivative is present in an amount of at least 20 parts. If the content of an acid derivative with a stronger acidity than the carboxylic acid functional polymer in Part B exceeds 80 parts the viscosity of the mixture became too high for optimal handling of the dental composition. For acetic acid which was used for the comparative examples 44-47, no such gel formation was seen.

### Example 48/Comparative Example 49

For solution A, solution B3 and solution B45 and the mixture of one part of solution A and four parts of each of solutions B3 and B45, the viscosity versus shear rate was determined as described above. The results are shown in Table 2. While solution A, B3 and B45 exhibited Newtonian behavior, the mixture of A and B3 revealed shear thinning. At low shear rates, the mixture of solution A and B3 showed a higher viscosity than the each of the solutions.

This example demonstrates that upon mixing of the two solutions of lower viscosity according to the present invention, a composition with a higher viscosity is obtained that exhibits shear thinning. This is very beneficial for the use in the dental field, because dosing and mixing of the solutions is facilitated, pooling of the mixture is avoided, and a smooth, homogeneous film is obtained. Inversely, for the comparative example no. 49 with the mixture of solution A and B45 no such shear thinning was found.

TABLE 2

| Example | | | | | | |
|---|---|---|---|---|---|---|
| no. | shear rate [s⁻¹] | 0.5 | 1 | 10 | 100 | 1000 |
| 48 | viscosity [Pa · s] of A | 0.3 | 0.2 | 0.2 | 0.2 | 0.2 |
| | viscosity [Pa · s] of B3 | 0.9 | 0.9 | 0.9 | 0.9 | 0.9 |
| | viscosity [Pa · s] of mixture A, | 1.4 | 1.2 | 0.7 | 0.4 | 0.4 |
| | B3 | | | | | |
| 49 comp. | viscosity [Pa · s] of A | 0.3 | 0.2 | 0.2 | 0.2 | 0.2 |
| | viscosity [Pa · s] of B45 comp. | 0.2 | 0.2 | 0.2 | 0.2 | 0.2 |
| | viscosity [Pa · s] of mixture A, | 0.2 | 0.2 | 0.2 | 0.2 | 0.2 |
| | B45 | | | | | |

### Example 50

For solution A and solution B26 and for the mixture of one part of solution A and four parts of solution B26, the viscosity versus shear rate was determined as described above. The results are shown in Table 3. While solution A and B26 exhibited Newtonian behavior, the mixture of A and B26 revealed shear thinning while the shear rate was ramped up. At low shear rates, the mixture showed a strong increase in viscosity with respect to solutions A and B 26. For the decreasing shear rate direction, an increase in the viscosity was observed indicating at lease partly reversible shear thinning.

This example also demonstrates that upon mixing of two solutions of lower viscosity according to the present invention, a composition with a higher viscosity is obtained that exhibits shear thinning.

TABLE 3

| Example | | | | | | |
|---|---|---|---|---|---|---|
| no. | shear rate [s⁻¹] | 0.5 | 1 | 10 | 100 | 1000 |
| 50 | viscosity [Pa · s] of A | 0.3 | 0.2 | 0.2 | 0.2 | 0.2 |
| | viscosity [Pa · s] of B26 | 1.3 | 1.3 | 1.3 | 1.3 | 1.3 |
| | viscosity [Pa · s] of mixture A, | 8.3 | 6.0 | 2.5 | 1.1 | 0.8 |
| | B26 | | | | | |

### Examples 51-58

Adhesive securing of a methacrylate based composite restorative material (FILTEK Z250) on enamel and dentin of bovine teeth was performed as described above for the 1:4 mixtures of solution A and solutions B3, B6, B13, B16, B26, B29, B36, and B39. The results can be found in Table 4.

TABLE 4

| | | adhesion on | adhesion on |
|---|---|---|---|
| Example no. | mixture A, Bx | enamel [MPa] | dentin [MPa] |
| 51 | A:B3 | 10.7 | 11.1 |
| 52 | A:B6 | 14.7 | 15.4 |
| 53 | A:B13 | 15.1 | 16.0 |
| 54 | A:B16 | 15.2 | 8.2 |
| 55 | A:B26 | 0.1 | 0.5 |
| 56 | A:B29 | 0.1 | 0.0 |
| 57 | A:B36 | 1.7 | 6.7 |
| 58 | A:B39 | 3.3 | 3.8 |

### Example 59

At a ratio of 1:4, solution A was mixed with a solution C consisting of PGDMA (50 parts), PM2 (25 parts), TEGDMA (16.725 parts), PUMA (4.900 parts) CPQ (1.250 parts), EDMAB (2.000 parts), BHT (0.125 parts).

For this composition, mixing of solutions A and C was especially easy, although a strong increase in viscosity was obtained for the mixture. Thus, a preferred embodiment of the invention contains additionally at least one urethane functionalized compound.

The viscosity versus shear rate was determined as described above. The results are shown in Table 5. Both solutions A and C exhibited Newtonian behavior, whereas the mixture of A and C exhibited strong shear thinning. For the decreasing shear rate direction, an increase in the viscosity was observed indicating at least partly reversible shear thinning.

TABLE 5

| Example | | | | | | |
|---|---|---|---|---|---|---|
| no. | shear rate [s⁻¹] | 0.5 | 1 | 10 | 100 | 1000 |
| 59 | viscosity [Pa · s] of A | 0.3 | 0.2 | 0.2 | 0.2 | 0.2 |
| | viscosity [Pa · s] of C | 0.3 | 0.3 | 0.3 | 0.3 | 0.3 |
| | viscosity [Pa · s] of mixture | 6.0 | 4.1 | 0.5 | 0.2 | 0.2 |
| | A, C | | | | | |

### Examples 60-61

Adhesive securing of a methacrylate based composite restorative material (example 60; FILTEK Z250) and of an epoxy based filling material (example 61) as described in WO 01/10388, page 12, was performed with the composition from example 59 according to the method described above. The adhesion values for enamel and dentin are given in Table 6. For this mixture, on all substrates, a smooth homogeneous film was easily obtained.

TABLE 6

| | | adhesion on | adhesion on |
|---|---|---|---|
| Example | | enamel | dentin |
| no. | | [MPa] | [MPa] |
| 60 | Z250 | 13.0 | 11.0 |
| 61 | epoxy based filling | 14.1 | 12.1 |
| | material | | |

### Example 62

A solution D consisting of HEMA (30 parts), water (30 parts), a 1:1 copolymer of acrylic and maleic acid with an average molecular weight Mw=12000 (30.1 parts), DPIPF6 (4.9 parts), Brij 35 (4.9 parts), BHT (0.1 parts) was mixed at a ratio of 1:4 with solution C.

This composition exhibited very little gel formation upon mixing of solutions D and C, although a very strong increase in viscosity was obtained for the mixture with respect to solutions D and C. The viscosity versus shear rate was determined as described above. The results are shown in Table 7. While solutions D and C exhibited Newtonian behavior, the mixture showed strong shear thinning. For the decreasing shear rate direction, an increase in the viscosity was observed indicating at least partly reversible shear thinning.

TABLE 7

| Example | | | | | | |
|---|---|---|---|---|---|---|
| no. | shear rate [s⁻¹] | 0.5 | 1 | 10 | 100 | 1000 |
| 62 | viscosity [Pa · s] of D | 0.4 | 0.4 | 0.4 | 0.4 | 0.4 |
| | viscosity [Pa · s] of C | 0.3 | 0.3 | 0.3 | 0.3 | 0.3 |
| | viscosity [Pa · s] of mixture | 13 | 3.6 | 0.4 | 0.2 | 0.2 |
| | D, C | | | | | |

### Example 63

Adhesive securing of an epoxy based filling material as described in WO 01/10388, page 12, was performed with the composition from example 62 according to the method described above. The adhesion values for enamel and dentin are given in Table 8. For the mixture of solutions D and C, on all substrates, a smooth homogeneous film was easily obtained.

TABLE 8

| Example | | | |
|---|---|---|---|
| no. | adhesion [MPa] | enamel | dentin |
| 63 | epoxy based filling material | 6.4 | 5.0 |

### Examples 64 to 68

As examples 64 to 65 and as comparative examples 66 to 68, mixtures of PGDMA (100 parts) and solution E were prepared. Solution E1-5 consists of BisEMA (amount according to Table 9), HEMA (amount according to Table 9), VBP (20 parts), water (amount according to Table 9), DPIPF6 (0.5 parts), CPQ (0.5 parts), EDMAB (0.5 parts).

The solutions E1-5 were mixed in a ratio of 1 g of solution E1-5 to 1 g of PGDMA. As a test for the handling properties and the viscosity of the compositions the resulting mixtures were checked visually for gel formation. The formation of a gel by mixing the two parts E1-5 and PGDMA of the dental composition indicates an increase in viscosity and improved handling properties. In Table 9, “−” denotes no visible gel formation, “+” denotes a visible gel formation.

As can be seen from the Table 9 for the comparative examples dental compositions containing above 10% of water do not exhibit a desired viscosity.

TABLE 9

| | | BisEMA | | | |
|---|---|---|---|---|---|
| | | in | HEMA in | water in | |
| | | solution E | solution E | solution E | viscosity |
| example no. | Solution E | (parts) | (parts) | (parts) | of B + E |
| 64 | E1 | 50 | 20 | 10 | + |
| 65 | E2 | 40 | 20 | 20 | + |
| 66 comp. | E3 | 30 | 20 | 30 | − |
| 67 comp. | E4 | 20 | 20 | 40 | − |
| 68 comp. | E5 | 10 | 20 | 50 | − |

1. A dental composition comprising:
(a) at least one carboxylic acid functional polymer,
(b) at least one acid derivative with stronger acidity than the carboxylic acid functional polymer (a), substituted with at least one polymerizable, ethylenically unsaturated group, and
(c) 0.1 to 10 weight % of water,
wherein the carboxylic acid functional polymer (a) and the acid derivative (b) are present at least in an amount effective that the dental composition exhibits shear thinning, and/or a viscosity of from 1.0 to 20 Pa·s when measured with a plate/plate geometry at a shear rate of 0.5 to 1 s⁻¹.

2. A dental composition according to claim 1, comprising:
(a) 1 to 30 weight % of at least one carboxylic acid functional polymer, substituted with at least one polymerizable, ethylenically unsaturated group,
(b) 10 to 80 weight % of at least one acid derivative with stronger acidity than the carboxylic acid functional polymer (a), substituted with at least one polymerizable, ethylenically unsaturated group,
(c) 0.1 to 10 weight % of water,
(d) 1 to 80 weight % of at least one unsaturated monomer and/or prepolymer, and
(e) 0.1 to 10 weight % of initiators, stabilizers.

3. A dental composition according to claim 1, comprising 0.1 to 6 weight % of water.

4. A dental composition according to claim 2, comprising 0.1 to 6 weight % of water.

5. A dental composition according to claim 1, wherein the viscosity of the composition measured at a shear rate of from 0.5 to 1 s⁻¹ is from 1.0 to 20 Pa·s and the viscosity at a shear rate of 1000 s⁻¹ is less than 1.0 Pa s.

6. A dental composition according to claim 1, wherein the viscosity of the composition measured at a shear rate of 1000 s⁻¹ is less than 30% of the viscosity at a shear rate of 0.5 s⁻¹.

7. A dental composition according to claim 1, wherein the composition does not comprise any particulate viscosity modifiers.

8. A dental composition according to claim 2, wherein the composition does not comprise any particulate viscosity modifiers.

9. A dental composition according to claim 5, wherein the composition does not comprise any particulate viscosity modifiers.

10. A dental composition according to claim 1, wherein the acid derivative (b) is selected from the group consisting of ethylenically unsaturated phosphoric acid esters, ethylenically unsaturated phosphoric acid diesters, ethylenically unsaturated sulfonic acid esters, or ethylenically unsaturated dicarboxylic acid monoesters, with a pKₐ of less than 4.75.

11. A dental composition according to claim 1, wherein the acid derivative (b) is selected from the group consisting of (meth)acryloyloxyalkyl-phosphate with the alkyl group having one to five carbon atoms, 5-(meth)acryloyloxy-3-oxa-pentyl phosphate, bis((meth)acryloyloxyalkyl) phosphate, bis(5-(meth)acryloyloxy-3-oxa-pentyl) phosphate, glycerol-di(meth)acrylate-phosphate, bis(glycerol-di(meth)acrylate) phosphate, (trimethylolpropane dimethacrylate) phosphate, bis(trimethylolpropane dimethacrylate) phosphate, pentaerythritol trimethacrylate phosphate, mono((meth)acryloyloxyalkyl)maleate, sulfoalkyl (meth)acrylate, 2-acrylamido-2-methyl-propanesulfonic acid, dodecylbenzene sulfonic acid, 2-naphthalene sulfonic acid, trifluoroacetic acid, or trifluoromethanesulfonic acid.

12. A dental composition according to claim 1, wherein the acid derivative (b) is selected from the group consisting of (i) a mixture of reaction products of 2-hydroxyethyl methacrylate with phosphorus pentoxide, (ii) a mixture of reaction products of glycerol di(methacrylate) with phosphorus pentoxide, (iii) 2-sulfoethyl methacrylate, or (iv) mono(methacryloyloxyethyl)maleate.

13. A dental composition according to claim 1, wherein the acid derivative (b) is selected from the group consisting of (i) a mixture of reaction products of 2-hydroxyethyl methacrylate with phosphorus pentoxide, or (ii) a mixture of reaction products of glycerol di(methacrylate) with phosphorus pentoxide.

14. A dental composition according to claim 1, wherein the carboxylic acid functional polymer (a) is selected from the group consisting of homopolymers and copolymers of unsaturated mono-, di- or tricarboxylic acids and/or their anhydrides.

15. A dental composition according to claim 1, wherein the carboxylic acid functional polymer (a) is selected from the group consisting of homopolymers of poly(meth)acrylic acid, and copolymers of (meth)acrylic and itaconic acid, (meth)acrylic and maleic acid, methyl vinyl ether and maleic anhydride or maleic acid, ethylene and maleic anhydride or maleic acid, and styrene and maleic anhydride or maleic acid, wherein each polymer has at least one acidic group substituted with a polymerizable ethylenically unsaturated group.

16. A dental composition according to claim 1, wherein the composition further comprises at least one urethane functionalized compound substituted with at least one polymerizable, ethylenically unsaturated group.

17. A dental composition according to claim 16 wherein the polymerizable, ethylenically unsaturated group of acid derivative (b) comprises an acrylate, methacrylate, acryl amide and/or methacryl amide group.

18. A dental composition, comprising at least two parts A and B wherein part A comprises:
(a) at least one carboxylic acid functional polymer, and
(aa) 0 to 10 weight % of water,
and wherein part B comprises:
(b) at least one acid derivative with stronger acidity than the carboxylic acid functional polymer (a), substituted with at least one polymerizable, ethylenically unsaturated group, and
(bb) less than 0.5 weight % of water,
wherein the carboxylic acid functional polymer (a) and the acid derivative (b) are present at least in an amount effective that the mixture of part A and part B exhibit shear thinning and/or a viscosity that is higher than that of each of the separate parts A and B, when measured with a plate/plate geometry, at a shear rate of 0.5 to 1 s⁻¹.

19. A dental composition according to claim 18 wherein the carboxylic acid functional polymer (a) and the acid derivative (b) are present at least in an amount effective that the mixture of part A and part B exhibits shear thinning, and/or a viscosity of at least 2 Pa·s at a shear rate of 0.5 s⁻¹.

20. The dental composition according to claim 1, wherein the acid derivative (b) is present in an amount of 20-80 parts by weight of the total composition.

21. The dental composition according to claim 1, wherein the amount of the acid derivative (b) and the carboxylic acid functional polymer (a) in the total composition is 20-90 parts by weight.

22. The dental composition according to claim 1, wherein the carboxylic acid functional polymer (a) is present in an amount of 2-15 parts by weight of the total composition.

23. The dental composition according to claim 1 having an adhesion to enamel and/or dentin in the range of 1-40 MPa.
